(19) United States
(12) Patent Application Publication
(10) Pub. No.: US 2014/0190029 A1
(43) Pub. Date: Jul. 10, 2014

(54) Life Milestone Recording Device and Method of Use

(71) Applicant: Terry J. Blakely, San Antonio, TX (US)

(72) Inventor: Terry J. Blakely, San Antonio, TX (US)

(21) Appl. No.: 14/151,764

(22) Filed: Jan. 9, 2014

Related U.S. Application Data
(60) Provisional application No. 61/750,778, filed on Jan. 9, 2013.

(51) Int. Cl. A61B 5/107

(52) U.S. Cl. CPC A61B 5/1072
USPC 33/512

(57) ABSTRACT

A life milestone recording device and method of use is disclosed herein which utilizes a growth board system that employs a means to attach and detach geometric objects as measurement points on the growth board. The geometric objects are utilized to record additional user indicia which correlate to the growth measurement. The life milestone recording device generally includes a measuring base member and a measurement recording member. The base member includes measuring indicia in both English and metric units and at least one attaching member for engaging the measurement recording member. The measurement recording member includes planar faces used to label with user indicia and at least one attaching member for engaging the base member.

### CROSS-REFERENCE TO RELATED APPLICATIONS

This application claims the benefit under Title 35 United States Code §119(e) of U.S. Provisional Patent Application Ser. No.: 61/750,778; Filed: Jan. 09, 2013, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates generally to a device and method of use for taking and recording growth measurements and correlating those measurements with other life milestone indicia. More specifically, the present invention relates to a device and method of use for recording growth measurements along with other life milestone indicia such as, but not limited to, a subject's weight, shoe size, notes, and photographs.

### BACKGROUND OF THE INVENTION

Without limiting the scope of the disclosed device and method, the background is described in connection with a novel device and approach to record life milestones of a subject.

The field's prior art reflects many approaches and devices in providing a means for maintaining growth charts and related indicia. This information is important to maintain for various reasons such as detecting abnormalities in the growth of a subject as well as comparing the trends and metrics with the other indicia maintained. In addition, it is important to parents and family members to have a recording and preservation of life milestone events of their children.

A first example of a growth chart device in the prior art is described in U.S. Pat. No. 7,475,487 issued on Jan. 13, 2009 to Dennis E. Johnson. In this example, the device is comprised of a height recording album containing journal pages with transparent pockets for inserting cards. In this prior art, the device maintains a limited number of pockets that are not adjustable. This provides the user a fixed amount of space for recording. In addition, this device utilizes a string mechanism for attaching the device to the door, an approach that is not secure and not easy to attach and detach from a door.

A second example of a growth chart device in the prior art is described in U.S. Pat. No. 7,059,060 issued on Jun. 13, 2006 to Daryl L. Baumgartner. In this example, the device is directed to a linear measuring apparatus having a side for measuring and a side for recording information. Here, there are no means for attaching the device to an object and the space for recording indicia is limited, permanent, and fixed to the linear device as well.

In view of the foregoing, it is apparent that there exists a need in the art for a life milestone recording device which overcomes, mitigates, or solves the above problems in the art. It is the purpose of this invention to fulfill this and other needs in the art which will become apparent to the skilled artisan once given the following disclosure.

### BRIEF SUMMARY OF THE INVENTION

The present invention, therefore, provides a device and method of use for a life milestone recording device. That is, a device that is capable of tracking the growth milestones of a subject from birth to adulthood by recording growth measurements and capturing moments in time and correlating those measurements with other indicia such as photographs and other measurements and notes.

In one embodiment, the life milestone recording device includes a measuring base member and a measurement recording member. The base member includes measuring indicia in both English and metric units and at least one attaching member for engaging the measurement recording member. The measurement recording member, in one embodiment, includes planar faces used to label with user indicia and at least one attaching member for engaging the base member.

In summary, the present invention discloses an improved device and method of use for a life milestone recording device. More specifically, the present invention relates to a device and method of use for recording growth measurements along with other life milestone indicia such as, but not limited to, a subject's weight, shoe size, note, and photographs.

### BRIEF DESCRIPTION OF THE SEVERAL VIEWS OF THE DRAWINGS

The accompanying drawings, which are incorporated in and form a part of the specification, illustrate a preferred embodiment of the present invention, and together with the description, serve to explain the principles of the invention. It is to be expressly understood that the drawings are for the purpose of illustration and description only and are not intended as a definition of the limits of the invention. In the drawings:

FIG. 1 is an exploded front perspective view of one example of the life milestone recording device in accordance with the teachings of the present disclosure;

FIG. 2 is a front perspective view of one example of the life milestone recording device illustrating a growth measurement being taken in accordance with the teachings of the present disclosure;

FIG. 3 is a front perspective view of one example of the life milestone recording device's geometric cube marker in accordance with the teachings of the present disclosure;

FIG. 4 is a front perspective view of one example of the life milestone recording device in accordance with the teachings of the present disclosure.

### DETAILED DESCRIPTION OF THE INVENTION

Disclosed herein is an improved device and method of use for a life milestone recording device. The numerous innovative teachings of the present invention will be described with particular reference to several embodiments (by way of example, and not of limitation).

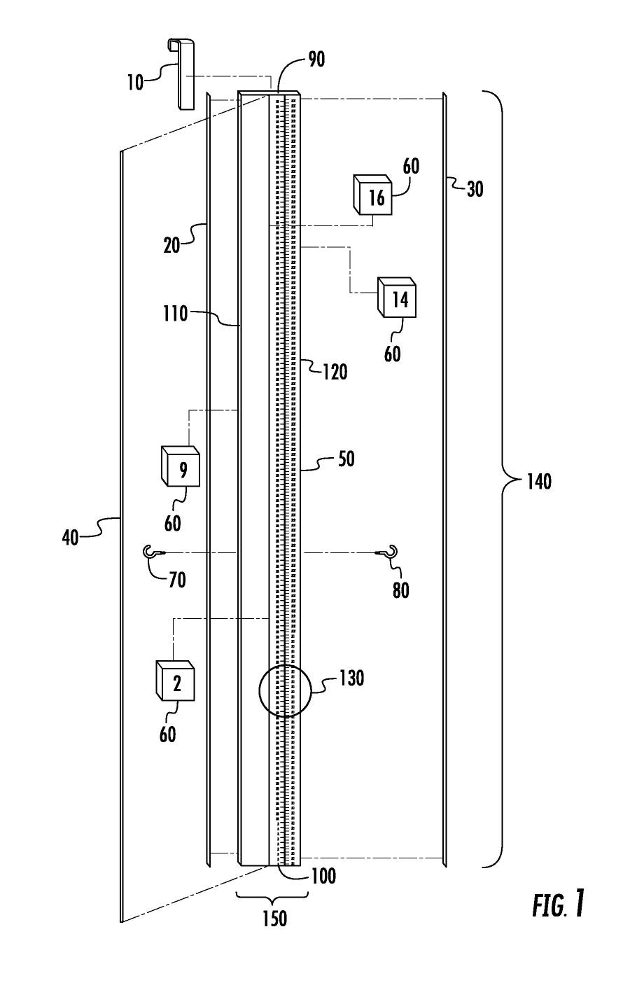

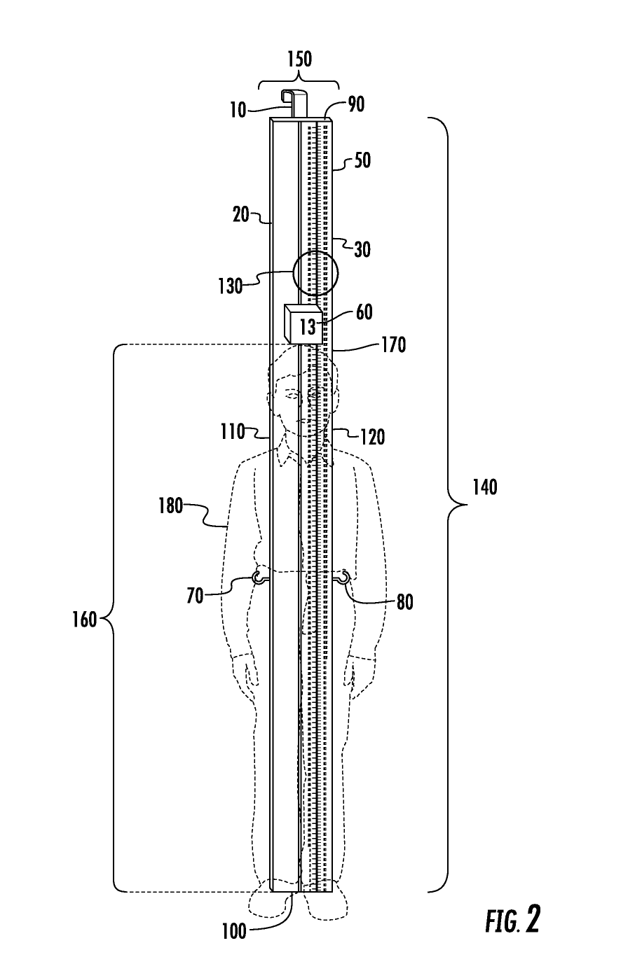

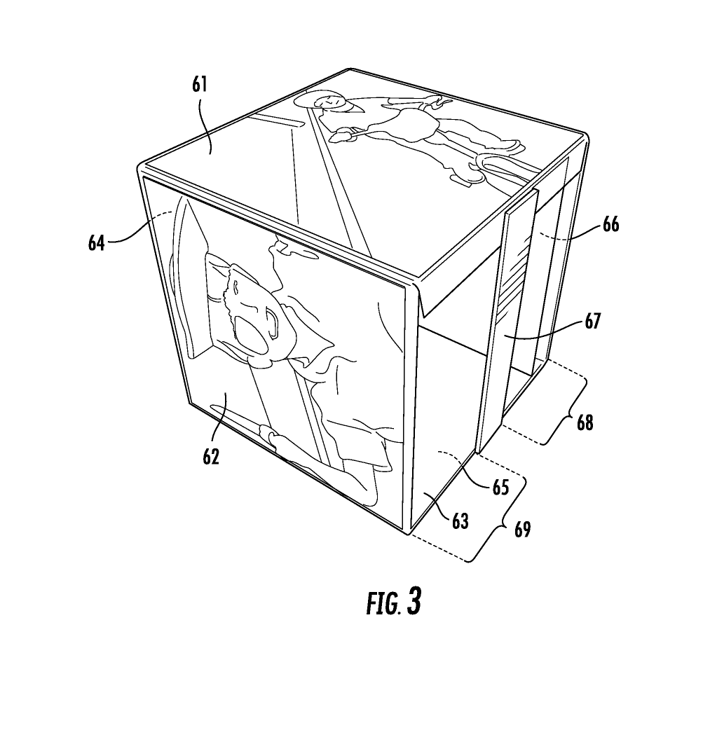

Reference is first made to FIG. 1, an exploded front perspective view of one embodiment of the life milestone recording device. In this illustration the components of the device are shown. The life milestone recording device generally includes a measuring base member 50 and at least one measurement recording member 60. The base member 50 in one embodiment is constructed out of rigid materials such as but not limited to polymers, wood, and or foam. In one preferred embodiment, the base member 50 has the dimensions of 6 inches width 150, 78 inches length 140, and 0.5 inches depth. The base member 50 includes length measuring indicia 130 on the front sides' right half running from bottom to top in both English and metric units and at least one attaching member 20, 30, 40 for engaging the measurement recording member 60. The length measuring indicia 130 allows users of the device to take height recording measurements. In an embodiment, the length measuring indicia 130 includes printed ruler markings from 0-78 inches, 0-6 feet 6 inches, and 0-197 centimeters running from bottom to top. The base member 50 also may include a connecting member 10 for attaching the base member 50 to an object such as a door. In one embodiment, the connecting member 10 is a metal bracket hangar attached to the back side of the base member 50. The connecting member 10 allows the base member 50 to be quickly attached or detached to the front or inside of a door. This allows the user to have the device visible (outside of a closet door) or stored away (inside a closet door) as they prefer. In a preferred embodiment, the base member's 50 attaching members 20, 30, 40 are 0.5 inch width and 78 inch length magnetic strip tape which run from the bottom to the top of the base member 50. Attaching member 20 is on the left side 110 of the base member 50. Attaching member 30 is on the right side 120 of the base member 50. Attaching member 40 is in the middle of the front side 110 of the base member 50. Other embodiments include the attaching members 20, 30, 40 being of various lengths shorter than the length of the base member 50 but having lengths sufficient enough to designate the measurement position 170 (FIG. 2) on the length measuring indicia 130 by attaching the measurement recording member 60. The base member 50 in a preferred embodiment may also include storing member attachment points 70, 80 for attaching items such as but not limited to drawstring bags and backpacks used to store the measurement recording members 60. The measurement recording member 60 includes geometric objects with planar faces used to label with user indicia and at least one measurement recording member attaching member 67 (FIG. 3) for engaging the base member's 50 attaching members 20, 30, and 40. In the illustration shown, the measurement recording members 60 are six sided clear photo boxes with the dimensions of 3 inches width, 3 inches length, and 3 inches depth. In a preferred embodiment, the measurement recording member's 60 attaching member is 0.5 inch width and 3 inch length magnetic strip tape which runs along the back face at several possible locations to be discussed more fully. The attaching members 20, 30, 40 of the base member 50 and the attaching member of the measurement recording member 60 allow the measurement recording members 60 to be quickly attached and detached at various locations along the base member's 50 length 140. This aspect of the invention allows the base member 50 to be used over and over and with multiple individuals.

Reference is next made to FIG. 2, a front perspective view of an embodiment of the life milestone recording device, illustrating a height measurement being taken. To obtain an individual's 180 height 160, the person will stand against the life milestone recording device with their back against the base member 50 and their feet together as illustrated in the figure. A measurement recording member 60, which is held to the base member's 50 attaching member 40 by magnetic forces, is slid down the attaching member 40 until the measurement recording member 60 rests on top of the individual's head. The individual then steps away from the base member 50 with the measurement recording member 60 maintaining the measurement position by 170 magnetic forces. Using the measurement position 170, which is the bottom of the measurement recording member 60, the height 160 may be obtained from the length measuring indicia 130. Also in this illustration, it can be seen that the measurement recording member 60 has been labeled with thirteen (13), the individuals' age.

Reference is now made to FIG. 3, a front perspective view of one example of the life milestone recording device's recording member 60. In this illustration, the measurement recording member 60 is a six sided cube having a top side face 61, a bottom side face 65, a left side face 66, a right side face 62, a front side face 64, and a back side face 63. In this illustration, the attaching member 67 of the measurement recording member 60 is attached to the middle of the back side face 63 having equal distances 68, 69 apart from the left side face 66 and the right side face 62. This position of the attaching member 67 is for cubes to be placed on the base member's 50 attaching member 40 which is on the front side of the base member 50. This location of the attaching member 67 will not work for the base member's 50 left side 110 and right side 120 attaching members 20, 30 due to the measurement recording member 60 hitting the object to which the base member 50 is attached to. For the base member's 50 left side 110 attaching members 20, the measurement recording member's 60 attaching member 67 will be located on the back side face 63 on the edge with the left side face 66. That is, the distance 68 of the attaching member 67 from the left side face 66 will be zero with the distance 69 of the attaching member 67 from the right side face 62 will be maximized. When the measurement recording member 60 is attached to base member's 50 left side 110 attaching member 20, the measurement recording members' 60 right side face 62 will face towards the front. For the base member's 50 right side 120 attaching members 30, the measurement recording member's 60 attaching member 67 will be located on the back side face 63 on the edge with the right side face 62. That is, the distance 69 of the attaching member 67 from the right side face 62 will be zero with the distance 68 of the attaching member 67 from the left side face 66 will be maximized. When the measurement recording member 60 is attached to base member's 50 right side 120 attaching member 30, the measurement recording members' 60 left side face 66 will face towards the front. Each face of the measurement recording member may have indicia such as but not limited to age, height, weight, shoe size, photographs, and notes attached internally (for transparent material) or externally to each face. For internal attachment, the measurement recording member 60 will have a means for opening up the object such as but not limited to unfolding of the object or a hatch face. This allows many indicia to be correlated to a measurement point for tracking purposes. The indicia may also be removed, changed, and or updated as need be.

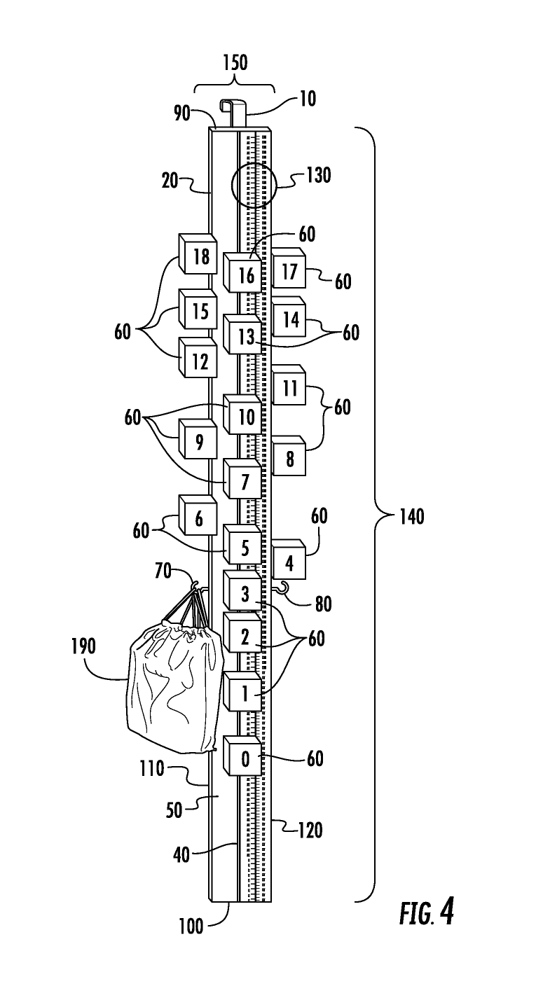

Reference is lastly made to FIG. 4, a front perspective view of an embodiment of the life milestone recording device. In this illustration the members locations are more readily seen. The attaching member's 20, 30, 40 locations on the base member 50 can be seen. In addition, the connecting member's 10 preferred location can be seen as well and is illustrated for connecting the base member 50 to a door. The connecting member 10 may be attached at various locations on the back of the base member 50 to allow for various door heights. That is, to maintain the proper measurement locations for the length measuring indicia 130 from the floor up, the connecting member 50 may be attached using various means at various height points on the back of the base member 50. In another embodiment, the back of the base member 50 is pre-drilled with two or more attachment holes at ½ inch increments to allow the connecting member 10 to be attached at those hole locations. In yet another embodiment, the length measuring indicia 130 may begin at the bottom of the base member 50 starting with the ½ inch marking to allow for a door's floor spacing at the bottom. Another aspect illustrated is the recording members 60 being attached to the base member 50. Illustrated are measurement recording members 60 with the year indicia (0-18) placed on the front face. 0 corresponds to the subjects' birth date, with 18 corresponding to year 18. This provides a visual representation of the growth indicia allowing easy tracking of the subject's development. This aids in the identification of any growth deficiencies. Another advantage of the device is to serve as an educational teaching tool by providing children or students with an interactive device for learning numbers, conversions between metric and English units, as well as taking measurements. In the teaching process, many games can be structured such as but not limited to proper measurement placement of the measurement recording members 60 and which subject can place all their cubes on the base member 50 the fastest. Also what can be seen in this illustration is the storing member attachment points 70, 80 with a drawstring bag 190 hung from attachment point 70. The drawstring bag is used to store the measurement recording members 60 and the drawstring bag 190 may be personalized with the subject's name by embroidering, stitching, or patches as a few examples. By incorporating this functionality in the device, multiple subject's can use the board at any given time. A number of bags could be hung from both sides of the device.

In brief, the present invention relates to a device and method of use for a life milestone recording device which allows the user to record growth measurements and correlate those measurements with other indicia such as weight, shoe size, and photographs.

The disclosed device and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method, except as may be outlined in the claims.

Alternative applications for this invention include using this device and method of use for obtaining other types of measurements and utilizing the measurement recording member as disclosed herein. Consequently, any embodiments comprising a one piece or multi piece device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device and/or methods disclosed and claimed herein can be made and executed without undue experimentation in light of the present disclosure. While the device and methods of this invention have been described in terms of preferred embodiments, it will be apparent to those skilled in the art that variations may be applied to the device and/or methods and in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A life milestone recording device comprising:
a measuring base member comprising length measuring indicia and an attaching member;
and at least one measurement recording member comprising an attaching member ;

2. The life milestone recording device of claim 1, wherein said measuring base member's length measuring indicia runs the length of said base member from bottom to top.

3. The life milestone recording device of claim 1, wherein said measuring base member's length measuring indicia runs the length of said base member from bottom to top on the front side.

4. The life milestone recording device of claim 1, wherein said measuring base member's length measuring indicia is in English and metric measuring units and runs the length of said base member from bottom to top on the front side.

5. The life milestone recording device of claim 1, wherein said measuring base member's length measuring indicia is in English and metric measuring units and runs the length of said base member from bottom to top on the front right half side.

6. The life milestone recording device of claim 1, wherein said measuring base member's length measuring indicia is in English and metric measuring units from zero to seventy eight inches and zero to one hundred ninety seven centimeters and runs the length of said base member from bottom to top on the front side.

7. The life milestone recording device of claim 1, wherein said measuring base member's attaching member runs the length of the left side, right side, and front of said measuring base member allowing attachment and detachment of said measurement recording member along said lengths.

8. The life milestone recording device of claim 1, wherein said measuring base member's attaching member is Velcro.

9. The life milestone recording device of claim 1, wherein said measuring base member's attaching member is a magnet.

10. The life milestone recording device of claim 1, wherein said measuring base member further comprises a connecting member.

11. The life milestone recording device of claim 1, wherein said connecting member is a metal bracket hangar attached to the top back side of said measuring base member.

12. The life milestone recording device of claim 1, wherein said measurement recording member's attaching member runs the length of the left side, right side, or back side of said measurement recording member allowing attachment and detachment of said measurement recording member with said measuring base member.

13. The life milestone recording device of claim 1, wherein said measurement recording member's attaching member is Velcro.

14. The life milestone recording device of claim 1, wherein said measurement recording member's attaching member is a magnet.

15. The life milestone recording device of claim 1, wherein said measurement recording member is a geometric shape having planar faces.

16. The life milestone recording device of claim 1, wherein said measurement recording member is a geometric shape having planar faces on which each planar face allows the recording of life milestone indicia.

17. The life milestone recording device of claim 1, wherein said measurement recording member is a six sided cube.

18. The life milestone recording device of claim 1, wherein said measurement recording member is a six sided cube allowing the planar faces to have recorded on them life milestone indicia.

19. The life milestone recording device of claim 1, further comprising a storing member.

20. The life milestone recording device of claim 1, wherein said storing member is a drawstring bag.
